(19) United States
(12) Patent Application Publication
(10) Pub. No.: US 2017/0035942 A1
(43) Pub. Date: Feb. 9, 2017

(54) CORROSION RESISTANT ZN-MG ALLOY IMPLANT MATERIAL OF HIGH STRENGTH AND TOUGHNESS AND ABSORBABLE BY HUMAN BODY

(71) Applicant: XI'AN ADVANCED MEDICAL TECHNOLOGY CO., LTD., Xi'an, Shaanxi (CN)

(72) Inventors: Gongyao ZHOU, Xi'an (CN); Gongqi QU, Xi'an (CN); Haibo GONG, Xi'an (CN)

(73) Assignee: XI'AN ADVANCED MEDICAL TECHNOLOGY CO., LTD, Xi'an, Shaanxi (CN)

(21) Appl. No.: 15/126,728

(22) PCT Filed: Apr. 25, 2014
(86) PCT No.: PCT/CN2014/076275

§ 371 Date: Sep. 16, 2016

(30) Foreign Application Priority Data
Mar. 19, 2014 (CN) 201410101607.9

(51) Int. Cl. A61L 31/02; C22C 18/00; A61L 27/58; A61L 31/14; A61L 27/04

(52) U.S. Cl. CPC A61L 31/022; A61L 31/148; A61L 27/047; A61L 27/58; C22C 18/00

(57) ABSTRACT

A corrosion resistant Zn—Mg alloy implant material of high strength and toughness and absorbable by the human body, the alloy implant material having: 96 wt % to 99.98% Zn and 0.002 wt % to 4% Mg. The alloy implant material is applied to absorbable medical implants, particularly vascular stents and orthopedic implants. The alloy implant material can be absorbed in the human body environment, avoiding pain to patients caused by secondary surgery, and has much better corrosion resistance than magnesium alloy, and being able to achieve the two indexes of high corrosion resistance and high strength and toughness.

### FIELD OF TECHNOLOGY

1. This invention relates to a fully degradable Zn—Mg alloy implant material which possesses corrosion resistance and high strength & toughness, this material belongs to the technical field of medical material.

### BACKGROUND TECHNOLOGIES

2. Intravascular stents are implantable wire mesh instruments for treatment of vascular diseases including coronary heart diseases. Its length is approximately 8 to 23 mm and its diameter is approximately 2.5 to 5 mm. Currently, the mainstream Drug Eluting Stent (DES) provides mechanical support for stenosis lesion blood vessels by expandable Co—Cr alloy wire mesh. The drug coating in the surface of the stent will release drug continuously for about a month, inhibiting growth of smooth muscle and lymphocytes and mitigate inflammation and immunoreactions. For a fairly long period, patients in China have very few information about this product due to the extremely low morbidity of coronary heart disease. With the improvement of national income and standards of living, as well as changes of dietary structure, China has seen an fast growing number of coronary heart disease cases. The rate of increase in coronary heart disease for males is 42.2% and that figure for females is 12.5%, and the patients group is getting younger. According to report of foreign medical community, patients of coronary heart disease must install intravascular stents when their conditions have developed to a certain stage to prevent myocardial infarction. After entering the 21^{st} Century, domestic stents surfaced and the number of patients who had stent implantations leaped. There were few patients of severe coronary heart disease implanted 7-8 stents in their blood vessels. According to one cardiovascular disease expert from Beijing Fuwai Hospital, total mortality caused by acute attacks of coronary heart disease exceeded one million per year in the past few years. Should these people have their intravascular stents implanted earlier, their mortality can be reduced by at least 50% Therefore, the full promotion of intravascular stents is a pressing concern.

3. Traditional intravascular stents are made of non-absorbable metals. There are two steps for manufacturing the stent. First, forge the metal ingots into tube stock through extrusion molding and then manufacture the tube stock into mesh stents by laser cutting technology. The shortcoming of traditional stents is that the metals are non-degradable and cannot extracted, the retained stent will eventually cause late thrombosis, In addition, laser cutting technology has a relatively low efficiency and thus the cost is high. Massive clinical cases validate the efficacy in lowering the stenosis reconstruction rate, however, after 1 to 5 years of implantation of this type of stents, the morbidity of thrombosis of patients will be as high as 3% to 9%, and more shockingly, the mortality after formation of thrombosis is as high as 30%. The problems about late thrombosis frequently occur in clinical cases is right the technical bottleneck that traditional stents cannot break through. The root cause is that the material for DES stents cannot degrade and absorbed by human body, instead, it will be left to the lesion blood vessel permanently. Lesion blood vessel only needs 1 to 3 months of mechanical support to restore blood vessel functionality. The stents left inside the blood vessels will pose a variety of side effects to the patients after three months.

4. Meanwhile, bone pegs and bone plates are commonly used medical implants to immobilize fractures and injured ligaments, particularly fractures near to joints or those extended to joints. The situation of population aging is becoming more and more severe in today's age, numbers of patients diagnosed with osteoporosis increases every year. Incidences such as car accidents and falling downs can often lead to comminuted fracture, During confrontational activities, fractures and ligament ruptures often happen to athletes.

5. Traditional bone pegs and bone plates industries are also experiencing the similar problems about intravascular stents where non-degradable metal cannot be extracted and a second operation is required, rendering great trauma to patients. At the same time, strength of traditional metal bone pegs and bone plates are way too high which can lead to stress shielding, stress shielding in turn will cause the injured bone structures difficult to regenerate and recover. Traditional polymers can absorb bone pegs and bone plates, therefore its strength is relatively low and some cases of it broke have been reported clinically.

6. Due to the various problems of traditional medical implants, medical material which can be absorbed by human body become the emphasis of research and development of the aforementioned fields (intravascular stent, orthopedic implants). Researchers conducted most profound researches on magnesium alloy in these years. Magnesium alloy can be absorbed by human body safely and has way higher strength and toughness than polymers, besides, the elasticity modulus of this material is closer to human bones. However, the corrosion resistance of magnesium is poor, it often degrades and being absorbed in human body in a very short period of time, thus unable to provide adequate mechanical support (service life).

### Technical Problems

7. For solving the above problems. this Invention provides a type of degradable Zn—Mg alloy implant material which can be absorbed by human body as well as absorbable medical implants made by this material, especially intravascular stents, bone pegs and bone plates. The corrosion resistant Zn—Mg alloy implant material with high strength and toughness of this Invention can accomplish three technical indexes, namely: can be absorbed by human body safely, appropriately mechanical strength and adequate mechanical support time. Absorbable stents made with this alloy material can not only effectively treat coronary heart disease, but also gives a possibility of bringing functions of blood vessels back to normal which cannot be done by traditional metal stents. Since there will be no metal implants being left permanently inside human body, blood vessels of patients treated with absorbable stents might acquire motor function, expansion and contraction as well as pulse, just like functional blood vessels without any treatment. Absorbable bone pegs and bone plates made by alloy material of this Invention can assist bone structures' regeneration and recovery. Their mechanical strength is matched with human's bone structures, thus there will not be any stress shielding or accidents of breakage.

### Technology Solutions

8. The composition and their weight percentages of Corrosion Resistant Zn—Mg Alloy

Implant Material of High Strength and Toughness and Absorbable by Human Body described in the technical scheme of this invention is: Zn 96-99.998 wt %, Mg 0.002-4 wt %.

9. Based on the composition of the above alloy material, we further optimize its composition to better its mechanical properties and biology corrosion performance as follows: Zn 97-99.995 wt %, Mg 0.005-3 wt %.

10. In order to acquire the best mechanical properties and biology corrosion performance, this invention strictly controls the content of impurities including Fe, Al, Mn etc.: purity of Zn equals to or greater than 99.95%; equals to or greater than 99.999% after optimization. Purity of Mg equals to or greater than 99.9%; equals to or greater than 99.99% after optimization. Total content of impurities excluding Zn and Mg is not greater than 0.5%, and not greater than 0.01% after optimization.

11. The alloy material produced in this Invention can be used to manufacture absorbable medical implants using routine method in the field. Preferably, the absorbable medical implants will be used to produce intravascular stents and orthopedic implants (eg. Bone pegs and bone plates).

### Beneficial Effects

12. Compared with existing technologies, the advantages of alloy material in this Invention are:

13. 1) It can be absorbed in human body environment, avoiding the pains brought to the patient by second operation.

14. 2) Corrosion resistance is way higher than magnesium alloy. The rate of degradation significantly reduced, allowing it to provide mechanical support for a longer period of time, preventing earlier failure of intravascular stent and bone pegs.

15. 3) Its mechanical strength is way higher than polymers. This alloy material has high toughness and is easy to make, its elasticity modulus is well compatible with human body.

16. 4) Indexes of high corrosion resistance and high strength & toughness can be accomplished simultaneously.

### INVENTION EXAMPLES

### Example Methods of This Invention

17. The following is concrete examples of the invention, which is used for explaining the technical proposal needed to solve the technical problem in the application document. It is helpful to understand the invention for technician in the field But the realization of the technical scheme of invention is not limited to these examples.

18. Example 1

19. Adopting advanced smelting and processing technology using high vacuum electromagnetic casting furnace, which made furnace burden produce suspension while heating at same time This can not only avoid the crucible pollution, also realize uniform mixing of alloying elements, and finish casting under vacuum conditions for avoiding the oxidation of the alloy; the content of alloy materials include Zn (99 wt %), and Mg alloy (1 wt %). Subsequently, the casting process and heat treatment were done for greatly improving the toughness and strength.

20. Example 2

21. The alloy material contained Zn 99.5 wt % and Mg 0.5 wt %. Other steps keep same with example 1.

22. Example 3

23. The alloy material contained Zn 98 wt %, Mg 2 wt %. Other steps keep same with example 1.

24. Example 4

25. The alloy material which contained example 1-3 carry out the immersion test of simulated body fluid.

26. Implementation results are shown in Table 1.

27. Table 1 the results of immersion test for simulated body fluid.

TABLE 1

| Number | Experimental material | Degrading rate(mm/year) |
|---|---|---|
| 1 | Example 1 | 0.19 |
| 2 | Example 2 | 0.14 |
| 3 | Example 3 | 0.27 |
| 4 | Pure Mg | 3.53 |
| 5 | Mg alloy WE43 | 3.89 |

29. This implantation example study in vitro degradation mechanism and degradation properties of Zn—Mg alloy according to the standard test method of ASTM-G31-72.And we found the degradation of rate for Zn—Mg alloy is slow and controllable under the environment of simulated body fluid which simulate the body fluid at 37° C. The immersion test of simulated body fluid proved the rate of degradation for Zn—Mg alloy is far lower than pure Mg and Mg alloy WE43. The surface of Zn—Mg alloy forms protection layer with low solubility and the degradation rate is much lower than that of Mg alloy, the lowest rate is only 0.14 mm/year, which can ensure intravascular stent and provide radial support for more than 6 months for lesion vessel, the support time of mechanics for fixed and bone plates is half a year at least, Slow and even degradation process allows blood vessels to regenerate gradually, and to restore stretch and beat eventually, as healthy blood vessels.

30. Example 5

31. The alloy material which contained example 1-3 carry out the tensile strength test.

32. The tensile mechanical properties of Zn—Mg alloy bars were evaluated according to the test standard ASTM-EB-04, which found the elastic modulus of Zn—Mg alloy was about 80 Gpa, the lowest tensile strength was 220 Mpa, and the maximum amount was up to 340 Mpa. The specific elongation of highest strength for Zn—Mg alloy is 11%, the lowest strength for Zn—Mg alloy is up to 29%, which means that Zn—Mg alloy is inversely proportional to its strength and toughness. It shows that the strength and toughness of Zn—Mg alloy can be controlled and meet the requirements of different types of stent and structural design of implant in department of orthopedics. The study found that the elastic modulus of human skin bone was 3-20 Gpa, and the yield strength was 35-280 Mpa. The alloy prepared by the invention has a good match with mechanical proprieties of human cortical bone.

33. Finally, it should be noted that the above examples are only to illustrate the technical scheme of the utility model, instead of the restrictions on them; A detailed description of the utility model is carried out by referring to the example of the utility model, the general personnel in the field should understand that it can still modify the technical scheme mentioned in the previous examples or have an equivalent exchange for some of the technical features; and these modification or replacement do not use the essence of the corresponding technical scheme out of the spirit and scope of the technical scheme of the utility model.

1. Corrosion Resistant Zn—Mg Alloy Implant Material of High Strength and Toughness and Absorbable by Human Body, Zn 96-99 998 wt %, Mg 0.002-4 wt %.

2. The Corrosion Resistant Zn—Mg Alloy Implant Material of High Strength and Toughness and Absorbable by Human Body described in claim 1, the said alloy material includes: Zn 97-99.995 wt %, Mg 0.005-3 wt %.

3. The Corrosion Resistant Zn—Mg Alloy Implant Material of High Strength and Toughness and Absorbable by Human Body described in claim 1 having a purity of Zn equal to or more than 99.95%.

4. The Corrosion Resistant Zn—Mg Alloy Implant Material of High Strength and Toughness and Absorbable by Human Body described in claim 3 having a purity of Zn equal to or more than 99.999%.

5. The Corrosion Resistant Zn—Mg Alloy Implant Material of High Strength and Toughness and Absorbable by Human Body described in claim 1 having a purity of Mg equal to or more than 99.9%.

6. The Corrosion Resistant Zn—Mg Alloy Implant Material of High Strength and Toughness and Absorbable by Human Body described in claim 5 having a purity of Mg equal to or more than 99.99%.

7. The Corrosion Resistant Zn—Mg Alloy Implant Material of High Strength and Toughness and Absorbable by Human Body described in claim 1, wherein total content of impurities other than Zn and Mg is not greater than 0.5%.

8. The alloy material mentioned in claim 1 is used for production of absorbable medical implants.

9. The alloy material described in claim 8, wherein the said absorbable medical implants are intravascular stents or orthopedic implants.

10. The Corrosion Resistant Zn—Mg Alloy Implant Material of High Strength and Toughness and Absorbable by Human Body described in claim 2 having a purity of Zn equal to or more than 99.95%.

11. The Corrosion Resistant Zn—Mg Alloy Implant Material of High Strength and Toughness and Absorbable by Human Body described in claim 10 having a purity of Zn equal to or more than 99.999%.

12. The Corrosion Resistant Zn—Mg Alloy Implant Material of High Strength and Toughness and Absorbable by Human Body described in claim 2 having a purity of Mg equal to or more than 99.9%.

13. The Corrosion Resistant Zn—Mg Alloy Implant Material of High Strength and Toughness and Absorbable by Human Body described in claim 12 having a purity of Mg equal to or more than 99.99%.

14. The Corrosion Resistant Zn—Mg Alloy Implant Material of High Strength and Toughness and Absorbable by Human Body described in claim 2, wherein total content of impurities other than Zn and Mg is not greater than 0.5%.

15. The alloy material mentioned in claim 2 is used for production of absorbable medical implants.

16. The alloy material described in claim 15 wherein the said absorbable medical implants are intravascular stents or orthopedic implants.
